(19) United States
(12) Patent Application Publication
(10) Pub. No.: US 2017/0056338 A1
(43) Pub. Date: Mar. 2, 2017

(54) Chocolate with antioxidants

(71) Applicants: Herbert PARDELL, Coral Springs, FL (US); Mark J. PASULA, Boynton Beach, FL (US); Paul J. MIANO, Deerfield Beach, FL (US)

(72) Inventors: Herbert PARDELL, Coral Springs, FL (US); Mark J. PASULA, Boynton Beach, FL (US); Paul J. MIANO, Deerfield Beach, FL (US)

(21) Appl. No.: 14/840,718

(22) Filed: Aug. 31, 2015

(51) Int. Cl. A61K 31/122; A23G 1/56; A23G 1/42; A23G 1/48; A61K 36/28; A61K 31/12; A61K 9/08; A61K 9/00; A61K 9/14; A61K 31/355; A61K 31/194; A61K 31/015; A61K 31/375; A61K 31/593; A23G 1/32

(52) U.S. Cl. CPC A61K 31/122; A23G 1/32; A23G 1/56; A23G 1/426; A23G 1/48; A23G 1/423; A61K 36/28; A61K 31/12; A61K 9/08; A61K 9/0095; A61K 9/14; A61K 31/355; A61K 31/194; A61K 31/015; A61K 31/375; A61K 31/593; A23V 2002/00

(57) ABSTRACT

A chocolate mixture comprising chocolate, preferably dark chocolate and an antioxidant mixture which includes:

| Ingredient | Minimum | | Maximum | |
|---|---|---|---|---|
| Betacarotene | 2000 | IU | 6000 | IU |
| Vitamin E Succinate 1185 IU/g | 200 | IU | 400 | IU |
| Vitamin C | 100 | mg | 400 | mg |
| Vitamin D3 | 200 | IU | 400 | IU |
| Vitamin K1 | 20 | μg | 80 | μg |
| Coenzyme Q10 | 20 | mg | 100 | mg |
| CURCUMAX ™ Curcumin Extract | 100 | mg | 300 | mg |
| in Oil | | | | |

The antioxidant mixture makes up from about 4% to about 8% of the weight of the combination of chocolate and mixture. Other additives, such as fruit, nuts and candies may also be added, but their weight is not factored into the calculation of the relative proportion of chocolate to antioxidant mixture.

### BACKGROUND OF THE INVENTION

1. Field of the Invention

The invention is directed to chocolate, and, in particular, to a new and improved chocolate with antioxidants which is both tasty and more healthful than heretofore known, as well as to a method for making such an improved chocolate.

2. Description of the Related Art

Chocolate is one of the most popular foods in the world, prized for its smooth texture and rich flavor. It is eaten and enjoyed everywhere.

There are three basic kinds of chocolate, dark (also known as bittersweet or semi-sweet), milk and “white”. Dark chocolate, i.e., chocolate which contains at least 65% cacao, is known to convey certain health benefits in addition to its pleasing taste. For example, it is known to lower blood pressure, and the risk of heart disease. Counterintuitively, perhaps, some research suggests it may also lower the risk of Type II diabetes, because it increases insulin sensitivity. It is also a known stress reliever and mood enhancer.

One of the most widely accepted health benefits of dark chocolate is in improving heart health due to its having flavonoids, which are known antioxidants. Antioxidants are known to neutralize free radicals which are linked to various health issues. Still, like all foods, chocolate is best consumed in moderation, since edible chocolate also contains some fats and sugars which are not normally beneficial.

Given the prevalence of chocolate in common diets, and its quasi-addictive taste, there is a need to increase the health benefits of chocolate, to permit chocolate's consumers to maximize health benefits realized by consuming chocolate while not increasing the unfavorable downsides to eating chocolate.

In the past, there have been attempts to introduce various additions to chocolate, but not all have been accepted.

A concern with adding anything to chocolate is there is always a risk that the addition will upset the delicate balance among the health benefits, the taste and the texture of the resultant product, because adding health benefits to chocolate will not be of any use if the final product does not taste good or if it is unpleasant to eat.

Accordingly, there is a need for an improved chocolate which has enhanced health benefits, with a satisfying taste and a pleasant texture.

### SUMMARY OF THE INVENTION

It is an object of the invention is to provide an improved chocolate which can provide even greater health benefits without adding significant downside risks to eating chocolate, or impairing the taste of the chocolate itself which would ruin any potential health benefits which derive from eating it.

It is a further object of the invention to provide an improved chocolate which has a pleasing texture when being consumed, as well as a pleasing flavor.

Other objects and features of the present invention will become apparent from the following detailed description of the presently preferred embodiments.

### DETAILED DESCRIPTION OF THE PRESENTLY PREFERRED EMBODIMENTS

The basics of chocolate-making are well-known and well-practiced in the art. A basic starting point is to take an amount of raw chocolate and add sugar or other sweetener to the naturally bitter raw chocolate, together with some cocoa butter. The proportions of these, and other ingredients, vary, depending upon the nature of the desired resultant product. These processes are not changed to incorporate the inventive method or mixture, and so are mentioned only in passing.

Once a user has determined what type of chocolate is desired, it must be determined how much chocolate is desired to be produced at the end of the process. No basic differences in the processes described result from the scale of the manufacturing operations. The user chooses how much of the chocolate is to be produced and adds an amount of the inventive antioxidant mixture to the desired base of chocolate. The antioxidant is to be prepared in accordance with the following table, showing the relative proportions of the various ingredients of the inventive antioxidant mixture. By “relative proportions” it is meant that the various components listed are to be present in the proportions indicated in the table, even if the total weight of the antioxidant mixture is more or less than the total weights represented in the table. By way of example, a supply of the antioxidant mixture weighing only one-half of the amount of the weight represented by the table could be prepared by including betacarotene in the range of from about 1000 IU to about 3000 IU, Vitamin E Succinate in the range of from about 100 IU to about 200 IU, etc. It will also be appreciated by those of ordinary skill in the art that the boundaries expressed below are approximate, so that small deviations therefrom could be implemented. Additionally, it is intended that the expressed ranges are not to be interpreted as requiring anything other than the amounts described. For example, it is not intended that use of an amount of Vitamin C at the lower end of the expressed range would require that the other components would also have to be employed at the lower ends of their respective ranges. By way of specific example, use of 20 mg of coenzyme Q10 would not require the use of 200 IU of Vitamin D, which could be used, instead, in the amount of 400 mg.

The preferred ranges of the ingredients are:

| Ingredient | Minimum | | Maximum | |
|---|---|---|---|---|
| Betacarotene | 2000 | IU | 6000 | IU |
| Vitamin E Succinate 1185 IU/g | 200 | IU | 400 | IU |
| Vitamin C | 100 | mg | 400 | mg |
| Vitamin D3 | 200 | IU | 400 | IU |
| Vitamin K1 | 20 | μg | 80 | μg |
| Coenzyme Q10 | 20 | mg | 100 | mg |
| CURCUMAX ™ Curcumin Extract | 100 | mg | 300 | mg |
| in Oil | | | | |

All of the described ingredients are generally and widely available, except for the curcumin extract in oil, which is only available under the trademark CURCUMAX® from C-X Research, Inc. of Boynton Beach, Fla. It is a suspension of curcumin in sunflower oil, which is preferred over other types of oils for being low in monounsaturated fats and is well tolerated by the body. Other types of oil, however, may be used, such as olive oil, but these other types of oils may yield a product having an unpleasant taste or texture.

Chocolate made with this formula is made in accordance with well-known methods of manufacture of chocolate, from preparing the cocoa, to mixing the various components, to melting the chocolate and then cooling it to form the desired end product, with the only addition being the mixture of antioxidants listed above. In the preferred embodiment, the additive mixture comprises between about 4% and about 8% of the total weight of the end product, with the rest being traditional chocolate, preferably dark chocolate. It is preferred that the end product be formed by adding, or folding, the additive mixture into either melted of softened chocolate. After the chocolate mixture is fully mixed, then the mixture may be cooled into any desired form.

In accordance with known processes, the inventive chocolate mixture can be formed into bars or other types of chocolate, such as pieces or bite-sized candies, and may be further mixed with well-known and common additives, such as fruits, dried fruits, nuts or any other known additive. If other non-chocolate additives are added to the end product, the weight of those additives is not factored into the relative proportions of chocolate and additive.

The inventive chocolate may also be made into liquid form for pouring into a beverage, or in powder form for the same or other purpose.

In the preceding Detailed Description, reference was made to the accompanying drawings, which form a part of this disclosure, and in which are shown illustrative specific embodiments of the invention. In this regard, directional terminology, such as “top”, “bottom”, “left”, “right”, “front”, “back”, etc., is used with reference to the orientation of the FIGURE(S) with which such terms are used. Because components of embodiments can be positioned in a number of different orientations, the directional terminology is used for purposes of ease of understanding and illustration only and is not to be considered limiting.

Additionally, while there have been shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. An antioxidant mixture comprising, in relative proportions:
Betacarotene in the range of from about 2000 IU to about 6000 IU;
Vitamin E Succinate in the form of about 1185 IU/g, in the range of from about 200 IU to about 400 IU;
Vitamin C in the range of from about 100 mg to about 400 mg;
Vitamin D3 in the range of from about 200 IU to about 400 IU;
Vitamin K1 in the range of from about 20 μg to about 80 μg;
Coenzyme Q10 in the range of from about 20 mg to about 80 mg; and
a suspension of curcumin extract in oil in the range of from about 100 mg to about 300 mg.

2. A mixture comprising:
an amount of chocolate; and
an antioxidant mixture comprising, in relative proportions:
Betacarotene in the range of from about 2000 IU to about 6000 IU;
Vitamin E Succinate in the form of about 1185 IU/g, in the range of from about 200 IU to about 400 IU;
Vitamin C in the range of from about 100 mg to about 400 mg;
Vitamin D3 in the range of from about 200 IU to about 400 IU;
Vitamin K1 in the range of from about 20 μg to about 80 μg;
Coenzyme Q10 in the range of from about 20 mg to about 80 mg; and
a suspension of curcumin extract in oil in the range of from about 100 mg to about 300 mg;
wherein said antioxidant mixture weighs from about 4% to about 8% of the total weight of the chocolate and antioxidant mixture.

3. The mixture of claim 2, wherein said chocolate is dark chocolate.

4. The mixture of claim 2, wherein the mixture is formed into a bar of chocolate.

5. The mixture of claim 2, wherein the mixture further comprises an additive selected from the group consisting of: fruits, dried fruits, nuts, and candies.

6. The mixture of claim 2, wherein the mixture is in liquid form.

7. The mixture of claim 6, wherein the mixture is further mixed into a beverage.

8. The mixture of claim 2, wherein the mixture is in powder form.

9. A method of making a mixed chocolate comprising the steps of:
adding an antioxidant mixture to an amount of chocolate;
wherein said antioxidant mixture is formed of the following components, in relative proportion:
Betacarotene in the range of from about 2000 IU to about 6000 IU;
Vitamin E Succinate in the form of about 1185 IU/g, in the range of from about 200 IU to about 400 IU;
Vitamin C in the range of from about 100 mg to about 400 mg;
Vitamin D3 in the range of from about 200 IU to about 400 IU;
Vitamin K1 in the range of from about 20 μg to about 80 μg;
Coenzyme Q10 in the range of from about 20 mg to about 80 mg; and
a suspension of curcumin extract in oil in the range of from about 100 mg to about 300 mg;
whereby said antioxidant weighs in the range of from about 4% to about 8% of the weight of the mixed chocolate.

10. The method of claim 9, wherein said chocolate is dark chocolate.

11. The method of claim 9, further comprising the step of forming the mixed chocolate into a bar of chocolate.

12. The method of claim 9, wherein the method further comprises the step of adding to the mixed chocolate an additive selected from the group consisting of: fruits, dried fruits, nuts, and candies.

13. The method of claim 9, wherein the mixed chocolate is in liquid form.

14. The method of claim 13, further comprising the step of mixing the mixed chocolate into a beverage.

15. The method of claim 9, further comprising the step of melting said chocolate before adding said antioxidant mixture thereto.

16. The method of claim 15, further comprising the step of cooling the mixed chocolate after said antioxidant has been mixed thereinto.

17. The method of claim 16, further comprising the step of forming said chocolate into a bar of chocolate after said antioxidant has been mixed thereinto.

18. The method of claim 9, further comprising the step of softening the mixed chocolate before adding said antioxidant mixture thereto.

19. The method of claim 18, further comprising the step of cooling the mixed chocolate after said antioxidant has been mixed thereinto.

20. The method of claim 19, further comprising the step of forming the mixed chocolate into a bar of chocolate after said antioxidant has been mixed thereinto.
